(19) United States
(12) Patent Application Publication
(10) Pub. No.: US 2007/0219427 A1
(43) Pub. Date: Sep. 20, 2007

(54) Premium-Based Private Civil Security Policy Methods

(76) Inventor: Barrett H. Moore, Winnetka, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 11/466,953

(22) Filed: Aug. 24, 2006

Related U.S. Application Data
(63) Continuation-in-part of application No. 11/384,037, filed on Mar. 17, 2006.
Application No. 11/384,037 is a continuation-in-part of application No. 11/394,350, filed on Mar. 30, 2006.
Application No. 11/394,350 is a continuation-in-part of application No. 11/279,333, filed on Apr. 11, 2006.
Application No. 11/279,333 is a continuation-in-part of application No. 11/379,929, filed on Apr. 24, 2006.
Application No. 11/379,929 is a continuation-in-part of application No. 11/381,247, filed on May 2, 2006.
Application No. 11/381,247 is a continuation-in-part of application No. 11/381,257, filed on May 2, 2006.
Application No. 11/381,257 is a continuation-in-part of application No. 11/381,265, filed on May 2, 2006.
Application No. 11/381,265 is a continuation-in-part of application No. 11/381,277, filed on May 2, 2006.
Application No. 11/381,277 is a continuation-in-part of application No. 11/383,022, filed on May 12, 2006.
Application No. 11/383,022 is a continuation-in-part of application No. 11/420,594, filed on May 26, 2006.
Application No. 11/420,594 is a continuation-in-part of application No. 11/421,694, filed on Jun. 1, 2006.
Application No. 11/421,694 is a continuation-in-part of application No. 11/423,594, filed on Jun. 12, 2006.
Application No. 11/423,594 is a continuation-in-part of application No. 11/425,043, filed on Jun. 19, 2006.
Application No. 11/425,043 is a continuation-in-part of application No. 11/426,231, filed on Jun. 23, 2006.
Application No. 11/426,231 is a continuation-in-part of application No. 11/456,472, filed on Jul. 10, 2006.
Application No. 11/456,472 is a continuation-in-part of application No. 11/461,605, filed on Aug. 1, 2006.
Application No. 11/461,605 is a continuation-in-part of application No. 11/461,624, filed on Aug. 1, 2006.
Application No. 11/461,624 is a continuation-in-part of application No. 11/462,795, filed on Aug. 7, 2006.
Application No. 11/462,795 is a continuation-in-part of application No. 11/462,845, filed on Aug. 7, 2006.
Application No. 11/462,845 is a continuation-in-part of application No. 11/464,751, filed on Aug. 15, 2006.
Application No. 11/464,751 is a continuation-in-part of application No. 11/464,764, filed on Aug. 15, 2006.
Application No. 11/464,764 is a continuation-in-part of application No. 11/464,775, filed on Aug. 15, 2006.
Application No. 11/464,775 is a continuation-in-part of application No. 11/464,788, filed on Aug. 15, 2006.
Application No. 11/464,788 is a continuation-in-part of application No. 11/464,799, filed on Aug. 15, 2006.
Application No. 11/464,799 is a continuation-in-part of application No. 11/465,063, filed on Aug. 16, 2006.
Application No. 11/465,063 is a continuation-in-part of application No. 11/466,727, filed on Aug. 23, 2006.

(51) Int. Cl. A61B 5/00

(52) U.S. Cl. 600/300

(57) ABSTRACT

Prior to the occurrence of an applicable civilly-catastrophic event (108), survival resources comprising at least hydration and nutritional consumables are pre-positioned (101) by a first party. A second party then offers (102) premium-based private civil security policies to candidate policy holders with respect to providing civilly-catastrophic event-based access to these pre-positioned survival resources. When this second party receives (106) a premium for such a policy from a given policy holder, the authorized beneficiary as correspond to that policy are permitted (109) to access the pre-positioned survival resources in response to an applicable civilly-catastrophic event that occurs during a period of coverage as corresponds to that policy. The first party is then compensated (110) (for example, by the second party) with respect to such access.

### RELATED APPLICATIONS

This application comprises a continuation-in-part of:

SUBSCRIPTION-BASED PRIVATE CIVIL SECURITY FACILITATION METHOD as filed on Mar. 17, 2006 and having application Ser. No. 11/384,037;

SUBSCRIPTION-BASED CATASTROPHE-TRIGGERED MEDICAL SERVICES FACILITATION METHOD as filed on Mar. 30, 2006 and having application Ser. No. 11/394,350;

PERSONAL PROFILE-BASED PRIVATE CIVIL SECURITY SUBSCRIPTION METHOD as filed on Apr. 11, 2006 and having application Ser. No. 11/279,333;

RADIATION SHELTER KIT APPARATUS AND METHOD as filed on Apr. 24, 2006 and having application Ser. No. 11/379,929;

FRACTIONALLY-POSSESSED UNDERGROUND SHELTER METHOD AND APPARATUS as filed on May 2, 2006 and having application Ser. No. 11/381,247;

SUBSCRIPTION-BASED CATASTROPHE-TRIGGERED TRANSPORT SERVICES FACILITATION METHOD AND APPARATUS as filed on May 2, 2006 and having application Ser. No. 11/381,257;

SUBSCRIPTION-BASED MULTI-PERSON EMERGENCY SHELTER METHOD as filed on May 2, 2006 and having application Ser. No. 11/381,265;

SUBSCRIPTION-BASED CATASTROPHE-TRIGGERED RESCUE SERVICES FACILITATION METHOD AND APPARATUS as filed on May 2, 2006 and having application Ser. No. 11/381,277;

DOCUMENT-BASED CIVILLY-CATASTROPHIC EVENT PERSONAL ACTION GUIDE FACILITATION METHOD as filed on May 12, 2006 and having application Ser. No. 11/383,022;

RESCUE CONTAINER METHOD AND APPARATUS as filed on May 26, 2006 and having application Ser. No. 11/420,594;

PURCHASE OPTION-BASED EMERGENCY SUPPLIES PROVISIONING METHOD as filed on Jun. 1, 2006 and having application Ser. No. 11/421,694;

SUBSCRIPTION-BASED PRE-PROVISIONED TOWABLE UNIT FACILITATION METHOD as filed on Jun. 12, 2006 and having application Ser. No. 11/423,594;

RADIATION-BLOCKING BLADDER APPARATUS AND METHOD as filed on Jun. 19, 2006 and having application Ser. No. 11/425,043; and

PRIVATE CIVIL DEFENSE-THEMED TELEVISION BROADCASTING METHOD as filed on Jun. 23, 2006 and having application Ser. No. 11/426,231;

EMERGENCY SUPPLIES PRE-POSITIONING AND ACCESS CONTROL METHOD as filed on Jul. 10, 2006 and having application Ser. No. 11/456,472;

PRIVATE CIVIL DEFENSE-THEMED BROADCASTING METHOD as filed on Aug. 1, 2006 and having application Ser. No. 11/461,605; and

METHOD OF PROVIDING VARIABLE SUBSCRIPTION-BASED ACCESS TO AN EMERGENCY SHELTER as filed on Aug. 1, 2006 and having application Ser. No. 11/461,624;

SUBSCRIPTION-BASED INTERMEDIATE SHORT-TERM EMERGENCY SHELTER METHOD as filed on Aug. 7, 2006 and having application Ser. No. 11/462,795;

SUBSCRIPTION-BASED CATASTROPHE-TRIGGERED RESCUE SERVICES FACILITATION METHOD USING WIRELESS LOCATION INFORMATION as filed on Aug. 7, 2006 and having application Ser. No. 11/462,845;

PRIVATELY PROVISIONED SURVIVAL SUPPLIES DELIVERY METHOD as filed on Aug. 15, 2006 and having application number 11/464,751;

PRIVATELY PROVISIONED SURVIVAL SUPPLIES SUB-UNIT-BASED DELIVERY METHOD as filed on Aug. 15, 2006 and having application Ser. No. 11/464,764;

PRIVATELY PROVISIONED SURVIVAL SUPPLIES ACQUISITION METHOD as filed on Aug. 15, 2006 and having application Ser. No. 11/464,775;

PRIVATELY PROVISIONED SURVIVAL SUPPLIES CONTENT ACQUISITION METHOD as filed on Aug. 15, 2006 and having application Ser. No. 11/464,788;

METHOD TO PRIVATELY PROVISION SURVIVAL SUPPLIES THAT INCLUDE THIRD PARTY ITEMS as filed on Aug. 15, 2006 and having application Ser. No. 11/464,799;

WASTE DISPOSAL DEVICE as filed on Aug. 16, 2006 and having application Ser. No. 11/465,063;

SUBSCRIPTION-BASED PRIVATE CIVIL SECURITY RESOURCE CUSTOMIZATION METHOD as filed on Aug. 23, 2006 and having application Ser. No. 11/466,727;

the contents of each of which are fully incorporated herein by this reference.

### TECHNICAL FIELD

This invention relates generally to providing private civil security benefits.

### BACKGROUND

Many citizens of the world have long passed the point when a ready availability of the basic necessities of life is satisfactory in and of itself. Today's consumer-oriented citizens demand, and often receive, an incredibly diverse and seemingly ever-growing cornucopia of consuming and experiential options. Such riches are typically based, in turn, upon a highly interdependent series of foundational infrastructure elements. Examples of the latter include, but are certainly not limited to:

transportation infrastructure such as roads, bridges, railways, and so forth that facilitate the inexpensive and rapid movement of sometimes perishable goods from source to consumer;

communications infrastructure such as telephones, television, radio, and the Internet that facilitate the inexpensive and rapid sharing of news, advice, information, and entertainment; and

the totality of civil services such as police services, fire fighting services, medical services, and so forth that facilitate a sufficient degree of order and predictability to, in turn, permit the complex series of inter-related interactions that modern society requires in order to operate.

As powerful as the machinery of modern life appears, however, modern citizens are today perhaps more at risk of experiencing a serious disruption in their ability to prosper or even to survive en mass than is generally perceived. Providing the necessities of life in general requires a lot of things to all operate, more or less, correctly. To put it another way, a serious disruption to any significant element of civilized infrastructure can produce catastrophic results for a broad swatch of a given civil community. Any number of natural and/or non-naturally-caused events can sufficiently disrupt society's infrastructure and ability to provide one or more life-sustaining resources such as hydration, nutrition, shelter, and the like.

Many people believe and trust that their government (local, regional, and/or national) will provide for them in the event of such a civilly-catastrophic event. And, indeed, in the long view such is clearly a legitimate responsibility owed by any government to its citizens. That such is a consummation devoutly to be wished, however, does not necessarily make it so. Hurricane Katrina provided some insight into just how unprepared a series of tiered modern governmental entities may actually be to respond to even basic survival needs when a civilly-catastrophic event occurs.

Such insights, of course, are not particularly new. Civil preparedness shortcomings occasionally attract public attention and niche marketing opportunities exist with respect to provisioning the needs of so-called survivalists. Indeed, there are those who spend a considerable amount of their time and monetary resources attempting to ready themselves to personally survive a civilly-catastrophic event. Therein, however, lies something of a conundrum.

On the one hand, modern governments typically do little to proactively ensure the bulk survival (let alone the comfort) of their citizens in the face of most civilly-catastrophic events. On the other hand, attempting to take responsible actions to reasonably ensure one's own safety and security can become, in and of itself, nearly a full-time avocation and leave little time to actually enjoy the conveniences and opportunities of modern life. Such individual actions may even be frowned upon by the greater part of society which has grown accustomed and falsely secure with existing efficient just-in-time delivery systems that provide the illusion of plenty while undercutting the perception of risk.

As a result, many (if not most) individuals and their families are largely bereft of access to survival resources that they will need should a civilly-catastrophic event befall them. This shortcoming tends to be relatively comprehensive; most people have ready access to neither a sufficient selection of survival supplies nor a sufficient quantity. For people who do have a store of supplies set aside against such an eventuality, it can be a considerable burden to maintain and ensure the freshness, vitality, and usability of those supplies. At the same time, the same civilly-catastrophic event that occasions their need for supplies will also likely disrupt relevant supply chains enough to cause a partial or complete shortage of supplies at their local merchants. The unfortunate net result is a relatively near term severe need for a variety of survival supplies that will often go unmet for lengthy periods of time.

### BRIEF DESCRIPTION OF THE DRAWINGS

The above needs are at least partially met through provision of the premium-based private civil security policy methods described in the following detailed description, particularly when studied in conjunction with the drawings, wherein:

FIG. 1 comprises a flow diagram as configured in accordance with various embodiments of the invention;

FIG. 2 comprises a schematic view as configured in accordance with various embodiments of the invention; and

FIG. 3 comprises a flow diagram as configured in accordance with various embodiments of the invention.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION

Generally speaking, pursuant to these various embodiments, private civil defense benefits are provided. By one approach, prior to the occurrence of an applicable civilly-catastrophic event survival resources comprising at least hydration and nutritional consumables are pre-positioned by a first party. A second party then offers premium-based private civil security policies to candidate policy holders with respect to providing civilly-catastrophic event-based access to these pre-positioned survival resources. When this second party receives a premium for such a policy from a given policy holder, the authorized beneficiary (or beneficiaries) as correspond to that policy are permitted to access the pre-positioned survival resources in response to an applicable civilly-catastrophic event that occurs during a period of coverage as corresponds to that policy. The first party is then compensated (for example, by the second party) with respect to such access.

So configured, authorized beneficiaries of such premium-based private civil security policies will have concrete, predictable access to survival resources upon the occurrence (and/or threat) of a civilly-catastrophic event. The selection and quantity of emergency survival items can be generally selected (and their maintenance governed) by experts and hence relieve the authorized beneficiary of responsibility in this regard.

The aforementioned second party can comprise any of a wide variety of business entities. Serving in general as an underwriter for the civil defense benefits described, this second party can readily comprise, for example, an insurance underwriter, a financial products institution, and so forth. This approach, in turn, permits a large existing underwriting infrastructure to be leveraged in favor of vetting such benefits and advising and marketing to a large existing audience with respect to the value and availability of such civil defense benefits.

By one approach, if desired, one or more agents can be employed to assist with interacting with candidate policy holders and to arrange for candidate policy holders to become policy holders in fact. Such agents, for example, can be permitted to share in the distribution of received premiums as are proffered by policy holders. By one approach, this sharing can be structured to greatly favor an agent for an initial term of a new policy and to then retract to a lower maintenance level for follow-on subsequent terms as correspond to that policy.

These steps are readily facilitated without dependency upon governmental oversight, participation, or control (though in some embodiments it may be necessary to receive, for example, the approval of state-based insurance industry regulators). The particular supplies (and/or the quantity of supplies) provided can vary with the needs and requirements of the authorized beneficiaries. Importantly, via these teachings individuals can benefit from a greatly increased opportunity to bring a considerably improved measure of security into their lives, knowing that, should a civilly-catastrophic event indeed be visited upon them, they will have extraordinary and reliable access to survival supplies.

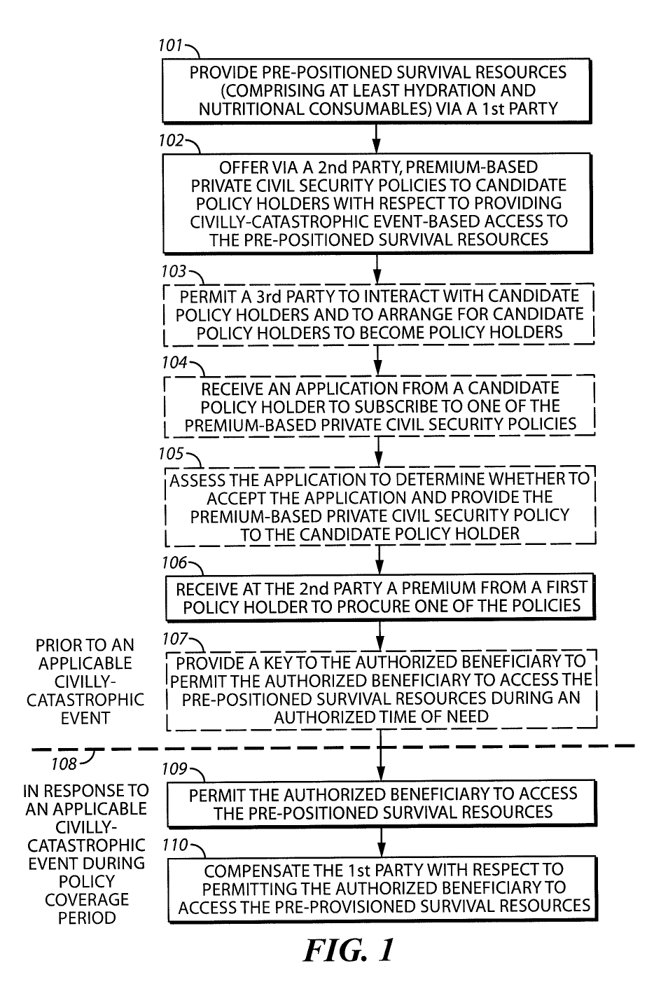

These and other benefits may become clearer upon making a thorough review and study of the following detailed description. Referring now to the drawings, and in particular to FIG. 1, a corresponding process provides for a number of actions to transpire prior the occurrence of an applicable civilly-catastrophic event. This can comprise, in part, providing 101, via a first party, pre-positioned survival resources comprising at least hydration and nutritional consumables. This first party can comprise, for example, a provider of private civil defense services to various parties under various approaches in addition to those set forth herein. By this approach, the first party therefore comprises an expert with respect to identifying, acquiring, aggregating, storing, and providing a variety of survival resources.

As noted, these survival resources can comprise hydration and nutritional consumables (such as, but not limited to, water and food). These pre-positioned survival resources can also encompass other assets and/or services. Examples include, but are not limited to, clothing, environmentally borne threat abatement (including, for example, personally worn items such as breathing masks, special clothing, and so forth), medical facilities, and so forth.

The private civil defense services of this first party can comprise other things as well, however. For example, this first party may also be involved with designing, providing, maintaining, and/or offering private civil defense shelters, private civil defense transport services, private civil defense rescue services, and/or the development and/or providing of privately developed customized instructions regarding appropriate survival actions to take in response to a civilly-catastrophic event. Numerous examples of such services are set forth in the various patent applications noted above. Accordingly, the present teachings will readily accommodate the pre-positioning of survival resources beyond the hydration and nutritional consumables noted. For example, these teachings will easily encompass the pre-positioning of private civil defense shelters, private civil defense transport assets and services, and so forth. Other possibilities exist as well including, but not limited to, private civil defense-based medical services, private civil defense-based communications assets and services, and so forth.

These survival resources can be pre-positioned in aggregated form at a single location if desired. Such a location might be provided, for example, within or near a densely populated urban area. By another approach, however, these survival resources can be pre-positioned at a plurality of geographically separated locations. The precise distance of separation can vary with the needs, requirements, and/or opportunities presented by a given application setting. In some cases it may be useful to provide such caches within only blocks of one another. In other cases it may be better to separate such caches by a few or even many miles. Such separation can serve, in some cases, to aid in ensuring that at least some of the pre-positioned survival resources will survive a civilly-catastrophic event with sufficient viability to merit the later distribution and/or usage of such resources. Such separation can also serve to ease the burden of facilitating access to these resources by authorized beneficiaries as described further below.

This process then provides for offering 102, via a second party, premium-based private civil security policies to candidate policy holders with respect to providing civilly-catastrophic event-based access to the pre-positioned survival resources. This second party can comprise a fully legally independent entity with respect to the first party mentioned above. If desired, however, this second party can be partially (though likely not fully) owned and/or controlled by the first party. In the latter case, for example, the second party may comprise a joint venture or partnership having the first party as a salient participant. As will be made clearer below, this second party comprises an underwriter. Those skilled in the art will understand that “underwriting” refers to the process that a large financial service provider (such as a bank, insurer, investment house, or the like) employs to assess the process of providing access to their products (such as equity capital, insurance, credit, and so forth) to a customer. In a very broad sense, underwriting involves the process of accepting the responsibility (and corresponding risk) of selling a particular allotment of some corresponding proposal, service, or undertaking. With this in mind (but without intending any particular limitation in this regard), suitable second parties might comprise such entities as insurance underwriters, financial products institutions, insurance brokers, banks, credit unions, academic institutions or clubs, a certified financial planner, a licensed securities dealer, a religious organization, a trade organization, or a fraternal organization to note but a few examples in this regard.

Such premium-based private civil security policies can be offered, if desired, directly from the second party to candidate policy holders. The present teachings will also readily accommodate, however, optionally permitting 103 a third party (including a plurality of third parties) other than the first and second party to interact with candidate policy holders and to arrange for candidate policy holders to become policy holders. There are numerous known legal and physical constructs to facilitate the establishment and use of such agents and brokers and other approaches in this regard are likely to be developed going forward. As such approaches are well known, and further as the present teachings are not particularly sensitive to the selection of any particular approach in this regard, for the sake of brevity further elaboration will not be presented here save where relevant to further points as are presented below.

The present teachings will also optionally accommodate receiving applications 104 from candidate policy holders seeking to subscribe to one of the aforementioned premium-based private civil security policies. Such an application can be initially received by the aforementioned second party or can, when the aforementioned third parties are employed to interact with candidate policy holders in this regard, be initially received by such third parties. In the latter case, the applications can then be forwarded on to the second party.

When employing a process that provides for receiving 104 such applications, these teachings will also optionally permit assessing 105 the applications to determine whether to accept a given application and thereby provide the premium-based private civil security policy to the corresponding policy holder. The aforementioned second party can perform this assessment 105 or all or part of the assessment process can be outsourced to another party as desired. The assessment can be based upon such criteria as may be of interest to the second party (and may include, or be supplemented by, the information provided in the application itself). In many cases, a primary point of inquiry in this regard will comprise assessing a risk that the candidate policy holder will actually need to seek access to the pre-positioned survival resources during a corresponding coverage period.

The risk factors themselves can vary with respect to the application setting as well as the risk tolerance and/or sensitivities of a given second party. Some exemplary risk factors of potential interest in this regard might include, but are not limited to, the authorized beneficiary's health, age, physical condition, relative personal physical mobility, survival training (as might be offered, for example, by the aforementioned first party), past behavior (as evidenced, for example, by credit records, criminal records, court records, and other privately maintained databases such as those employed by insurance companies to identify insurance fraud perpetrators or the like), and predicted future behavior (as may be predicated, for example, upon a profile as may be developed for a given authorized beneficiary using background investigative techniques, interviewing techniques, and the contents of the application itself).

This risk assessment, in turn, can serve to inform a decision regarding whether to provide the premium-based private civil security policy to the candidate policy holder and/or the amount of the premium to be required in exchange for providing such a policy. This risk assessment can also inform a decision to include, or exclude, particular civilly-catastrophic event risks and/or particular survival resources. As yet another example, such a risk assessment can lend guidance with respect to the duration of the corresponding coverage period and/or a corresponding benefits period as may pertain to the premium-based private civil security policy for a particular candidate policy holder.

In any event, this process also provides for receiving 106, at the second party, a premium from policy holders to procure corresponding policies as described above for one or more corresponding authorized beneficiaries. This premium may comprise any manner or combination of any form of consideration. Obvious candidates in this regard include money and other less-liquid forms of capital, investment, or economic interest. Such a premium may also comprise, in whole or in part, the rendering of services, materials, expertise, or the like.

When implementing this process in conjunction with the aforementioned third parties acting as agents/brokers, this step of receiving 106 the premium can further comprise, if desired, providing a commission to that third party. By one approach all or part of this commission can comprise at least some of the premium. The amount so provided can be fixed or vary with respect to one or more criteria or metrics of interest. For example, as an incentive to such agents to seek larger rather than smaller numbers of policy holders, the amount so provided can increase as the volume of policy holders that are attributable to a given agent/broker grows. Such growth can be aggregated on a continual basis, if desired, or can be constrained to results as are obtained between particular beginning and ending times.

It is also possible, if desired, to vary the amount so provided to such third parties as a function, at least in part, of whether a given premium has been received for a new policy holder or for an existing policy holder. For example, this can comprise providing a first relative portion of the premium to the third party with respect to a first (i.e., initial) term of a policy for a given one of the policy holders and a second, different relative portion (that is, for example, less than the first relative portion) of the premium to the third party with respect to a term of the policy that is subsequent to the first term for a given one of the policy holders. So practiced, the third parties may receive a relatively larger share of the premiums as are paid by a new policy holder than for premiums that correspond to an existing policy holder who seeks to continue (or expand) already existing protection.

If desired, such relative variations can be accommodated for any number of subsequent terms. For example, by one approach, differing relative portions of the premium can be provided to the third party with respect to at least some terms of the policy that are subsequent to the first term for a given one of the policy holders, where at least two of the differing relative portions of the premium are different from one another and from the first relative portion. By this approach, for example, the subsequent relative values can be incrementally reduced, or increased, over time as desired.

If desired, this process will also optionally accommodate providing 107 a key to authorized beneficiaries who have paid their premium to thereby facilitate and permit those authorized beneficiaries to access the pre-positioned survival resources during an authorized time of need. Such a key may comprise a physical item (such as, for example, an identification card or a physical lock key) and/or a non-physical item (such as, for example, a code such as a personal identification number, a password, or the like). When the key itself comprises a biometric characteristic as corresponds to the authorized beneficiary (such as, but not limited to, deoxyribonucleic acid (DNA), a retinal pattern, an asperity pattern (such as a fingerprint, a palm print, or the like), a voice print, a characteristic typing pattern, and so forth) this step can comprise (in conjunction with acquiring information, materials, and/or samples from the authorized beneficiary to thereby characterize the biometric of choice) providing instructions to the authorized beneficiary regarding presentation of such a biometric in order to gain the desired access. When the key comprises a code, by one approach this step can comprise providing the code to the authorized beneficiary. By another approach, this step can comprise receiving a code of choice from the authorized beneficiary and then providing confirmation to the authorized beneficiary regarding acceptance of that proffered code.

This process will also optionally accommodate delivering to the policy holders a short-term supply of survival resources comprising at least nutritional consumables. Hydration supplies can also be provided if desired. Such supplies can be provided, if desired, by the aforementioned first party. This short-term supply can comprise, for example, a sufficient quantity of survival resources to support survival of the corresponding policy holder for at least a predetermined number of days (such as, for example, a ten day period of time). This short-term supply can be provided in conjunction with the receipt of the above-described premium. In addition, or in lieu thereof, these teachings will also accommodate permitting such policy holders to purchase additional supply resources from the first party if they wish.

These teachings will further optionally accommodate delivering for the benefit of policy authorized beneficiaries a personal guide that is configured and arranged to provide information regarding specific personal actions to be uniquely taken by such persons in specific response to a civilly-catastrophic event having occurred. Such personal guides are well described in the previously mentioned and incorporated patent applications.

These teachings will readily accommodate individual policies of the type noted and/or a group policy approach. A group policy could operate in a similar manner to provide such services, under such conditions, for a corresponding group of authorized beneficiaries such as, but not limited to, a group of family members, the employees of a given enterprise, the members of a club, association, or some other shared affinity group, and so forth.

As noted, the above-noted steps are taken prior to an occurrence of an applicable civilly-catastrophic event. As such, they represent actions taken to better prepare the authorized beneficiaries to deal with a future prospective civilly-catastrophic event and not actions that are taken to assist persons who are presently afflicted by the effects of such an event. The existence of an applicable civilly-catastrophic event can be noted and/or confirmed by an appropriate party, such as the first or second party or even some third party of choice. If desired, such notice/confirmation can be published or otherwise made available to authorized beneficiaries and/or policy holders using any of a wide variety of means. For example, such a condition can be published at a corresponding website or via a particular audio or audio/visual broadcast (such as, for example, via civil defense television and/or radio broadcasting network programming). Such a condition might also be published by forwarding a corresponding email, instant message, Short Message Service (SMS) message or the like to such parties. It would also be possible, for example, to provide a telephone number which, when called, provides recorded and/or live information in this regard.

When and if an applicable civilly-catastrophic event (i.e., a civilly-catastrophic event of at least a predetermined category, kind, duration, nature, and/or level of impact) occurs 108 during the coverage period provided by the foregoing private civil security policies, these teachings then provide for permitting 109 the covered authorized beneficiaries to access the pre-positioned survival resources. If desired, this can comprise, in part, first (and/or secondly) that the authorized beneficiary is presently authorized to access these resources. By one approach, when using a key or other authentication mechanism of similar effect or nature, this can comprise using the key/mechanism to authenticate the authorized beneficiary's rights in this regard.

Such a determination can also, if desired, (in combination with a key/mechanism approach as described above or in lieu thereof) comprise monitoring corresponding circumstances as are relevant to a need for the authorized beneficiary to continue to have access to the pre-positioned survival resources. The particular circumstances monitored for this purpose can vary with the specifics of a given application setting. In many cases, for example, such circumstances will correlate with limitations regarding the provision of such relief in the premium-based private civil security policies. An illustrative but non-exhaustive listing of such circumstances can include, but is not limited to:
whether and to what extent the survival-challenging conditions as correspond to the civilly-catastrophic event continue to persist, worsen, or abate;
whether and to what extent publicly-sponsored survival assistance becomes (or remains) available;
specific limitations as are delimited by the policy as pertains to a given authorized beneficiary (regarding, for example, a specific term beyond which such benefits are no longer provided, a certain upper limit on the quantity of resources accessed or consumed, and so forth);
whether the authorized beneficiary has and continues to observe any rules as have been established by the first party (regarding, for example, behavior, cooperation, and so forth);
relevant declarations regarding disasterhood by applicable governmental authorities;
whether and to what extent a substantial lack of effective governmental influence exists with respect to an availability of survival requirements (which may be public or privately proffered);
a total number of people who are substantially adversely directly affected by the civilly-catastrophic event within an occurrence zone of the civilly-catastrophic event; and/or
confirmation (such as a declaration or a certification) by at least one of the first and second party that an applicable civilly-catastrophic event has occurred.

As noted earlier, there may be a plurality of locations where such pre-positioned survival resources are disposed. In such a case, such permission 109 may be further based upon whether a given authorized beneficiary has policy-based rights to only access a particular one of these locations or whether that authorized beneficiary has policy-based rights that permit access to more, or all, of such a plurality of pre-positioned survival resource locations.

This process then provides for compensating 110 the first party with respect to having permitted such authorized beneficiaries to access the pre-positioned survival resources. Such an action can be prompted, for example, by having the first party provide notice to the second party regarding the authorized beneficiary's access to the pre-provisioned survival supplies. Such notice can be provided on an individual basis or, if desired, can be submitted in some aggregated form. Such compensation may comprise a flat rate that covers all of the access activity of a given authorized beneficiary, a quasi-flat rate (as might correspond, for example, to a per diem-styled form of compensation), or an amount that reflects the actual usage the authorized beneficiary has made of the pre-positioned survival resources.

By one approach, this compensation 110 is provided directly to the first party by the second party. This would likely ease the burden that might be faced by a given authorized beneficiary to present an adequate payment during a time of need. If desired, however, this compensation 110 can be provided directly (in part or in whole) by the authorized beneficiary to the first party. In such a case the authorized beneficiary might then have a right (as per the terms of the premium-based private civil security policy) to seek full or partial reimbursement from the second party for such an expenditure.

Those skilled in the art will recognize and appreciate that the above described teachings can be applied and leveraged in a variety of ways to achieve a significant number of tangible and concrete benefits (including but not limited to facilitating a relatively higher degree of candidate policy holder awareness, a relatively higher level of resources availability, and/or a relatively more powerful set of motivations for various parties to adopt behaviors that in turn prompt an increased availability of potentially life-preserving resources for a relatively wide consuming audience than might likely be achieved in the absence of such teachings).

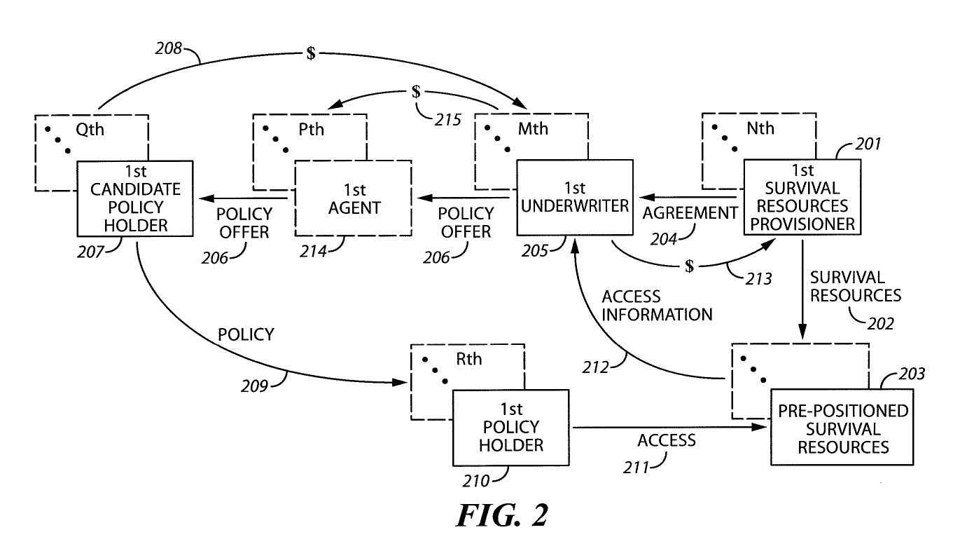

To provide a somewhat more specific illustrative example, and referring now to FIG. 2, a first survival resource provisioner 201 can provide survival resources 202 to one or more locations to thereby provide the aforementioned pre-positioned survival resources 203. If desired, a plurality of such provisioners 201 can optionally be accommodated as represented here by an Nth survival resource provisioner (where “N” will be understood to represent an integer greater than one).

The survival resources provisioner(s) 201 also has, for example, an understanding (via, for example, one or more corresponding agreements 204) with at least a first underwriter 205. As with the provisioner 201, there can also be a plurality of underwriters 205 (as represented here by an Mth underwriter (where “M” will be understood to represent an integer greater than one). This understanding can provide for the kinds of arrangements as are described herein. In particular, this understanding can establish the framework whereby the survival resource provisioner(s) 201 receives consideration in exchange for permitting certain parties as identified by the underwriter(s) 205 to access the pre-positioned survival resources 203.

The underwriter(s) 205 can then provide policy offers 206 to one or more candidate policy holders 207 (where the plurality of such candidate policy holders is represented by a Qth candidate policy holder (where “Q” will be understood to represent an integer greater than one)). These policy offers 206 can comprise, in accordance with these teachings, an offer to receive premium-based private civil security policies that, at least in part, permit the corresponding policy holder to access the aforementioned pre-provisioned survival resources 203 in response to an applicable civilly-catastrophic event.

By one approach, such policy offers 206 are extended without a middleman. By another approach, if desired, one or more intervening parties, such as one or more agents 214, can serve as a point of offer, information provisioning and/or acquisition, policy application preparation and submission, premium acceptance and/or forwarding, and so forth on behalf of the underwriter(s) 205. As noted, a plurality of such agents 214 can serve as described, where such a plurality is represented here by a Pth agent (where “P” comprises an integer greater than one).

To fully accept such a policy offer 206, a given candidate policy holder 207 submits a premium 208. This premium 208 will typically comprise money (or a corresponding negotiable instrument (such as a check) or transaction (such as a credit card transaction) but these teachings will also accommodate other forms of consideration such as, but not limited to, barter exchanges, services, option-based opportunities, and so forth. These teachings will also accommodate the offering of such a premium 208 as a single aggregated offering or as a plurality of offerings that are distributed, for example, over time. The amount of the premium can comprise a relatively fixed amount or can vary with time and or circumstances. By one approach, the premium for a new policy or a renewed, extended, and/or supplemental or replacement policy could vary with an assessment of such threats as may occasion a need to seek recovery under the policy. If desired, the amount of change in the premium for a given policy holder can be limited to a particular range of fluctuation on a term to term basis or as per some other temporal or circumstantial condition of interest.

This premium 208 may be supplied directly to the corresponding underwriter 205 or may, if desired, pass through the agent (or agents) 214 with whom the candidate policy holder 207 has been communicating. When one or more agents 214 has participated in effecting the transaction, these teachings will also accommodate providing some or all of that premium 208 to those agent(s) 214. As noted earlier, by one approach, a relatively larger portion of the premium can be provided to the agent(s) 214 for a new candidate policy holder 207 than might be provided for continuing (or expanding) coverage for an already-existing policy holder. These teachings will also accommodate providing other incentives of various kinds to the agents and/or the candidate policy holders to encourage their respective motivation, support, and/or interest with respect to the completion of such transactions.

In this way a given candidate policy holder 207 becomes the beneficiary of a private civil security policy 209 and hence becomes a policy holder 210. Again, there can be a plurality of such policy holders 210 (as represented here by an Rth policy holder where “R” comprises an integer greater than one). This status, in turn, permits the policy holder(s) 210 to gain access 211 to some or all of the pre-provisioned survival resources 203 as per the terms and conditions of their particular policy 209. When such access 211 occurs, the underwriter 205 receives corresponding access information 212 and uses that information to effect the provision of corresponding consideration 213 to the corresponding survival resources provisioner(s) 201. Such behavior can be directly accomplished by the underwriter 205 or can be partially or wholly accomplished by another party that the underwriter 205 engages for such a purpose.

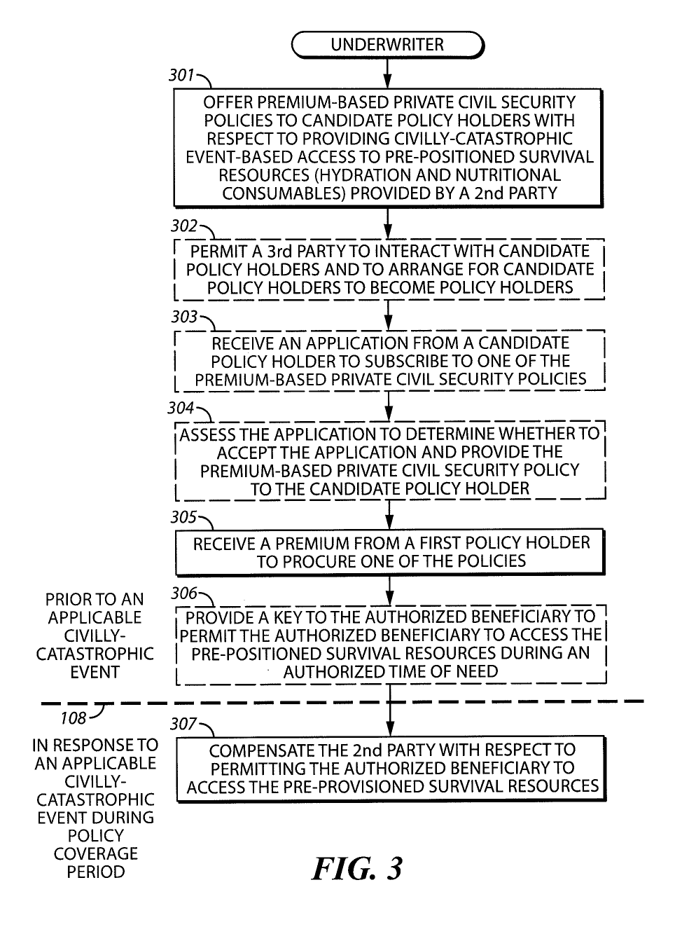

Referring now to FIG. 3, pursuant to these teachings a given underwriter is able to offer 301 premium-based private civil security policies to candidate policy holders with respect to the provision of civilly-catastrophic event-based access to pre-provisioned survival resources such as hydration and nutritional consumables as are provided by another party (such as the aforementioned survival resources provisioner(s)). This underwriter can interact directly with candidate policy holders in this regard or can permit 302, if desired, another party (or parties) to interact with candidate policy holders and to arrange for candidate policy holders to become policy holders.

By these teachings, if desired, this underwriter can optionally receive 303 applications from candidate policy holders who are seeking to subscribe to one of the premium-based private civil security policies and to assess 304 such applications to determine whether to accept the application and provide a premium-based private civil security policy to the candidate policy holder. In any event, such an underwriter can receive 305 a premium from a first policy holder who successfully procures such a policy and can then act to provide 306 (or to otherwise cause the provision of) a key to the authorized beneficiary of such a policy to permit that party to access the pre-provisioned survival resources during an authorized time of need.

When and if an applicable civilly-catastrophic event occurs during a given policy coverage period, that underwriter can then act to compensate 307 the party who provided the aforementioned pre-provisioned survival resources that were accessed by the policy holder.

These teachings permit existing business infrastructures and legacy-based relationships to be readily applied towards the offering and support of a completely new service; i.e., the provisioning of private civil security services and benefits. When offered through parties who are already experienced with the offering of other kinds of protective services and products (such as insurance, wealth protection instruments and plans, and so forth), one may reasonably expect that the offering of private civil security benefits will comprise a relatively natural fit and/or extension of such legacy offerings. This, in turn, can provide a powerful instrument to facilitate generating interest in a completely new kind of protective service and offering.

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept.

We claim:

1. A method of providing private civil defense benefits comprising:
prior to an applicable civilly-catastrophic event:
providing, via a first party, pre-positioned survival resources comprising at least hydration and nutritional consumables;
offering, via a second party, premium-based private civil security policies to policy holders with respect to providing civilly-catastrophic event-based access to the pre-positioned survival resources;
receiving, at the second party, a premium from a first policy holder to procure one of the policies for at least one corresponding authorized beneficiary;
in response to an applicable civilly-catastrophic event that occurs during a period of coverage as corresponds to the policy that is procured by the first policy holder:
permitting the authorized beneficiary to access the pre-positioned survival resources;
compensating the first party with respect to permitting the authorized beneficiary to access the pre-provisioned survival resources.

2. The method of claim 1 wherein the policies comprise at least one of:
time-limited rights of access;
event-limited rights of access;
inheritable rights of access;
rights of access predicated upon a series of periodic payments;
rights of access predicated upon a one-time payment;
ownership-based rights of access;
non-transferable rights of access;
transferable rights of access;
membership-based rights of access;
fractionally-based rights of access;
non-ownership-based rights of access;
option-based rights of access.

3. The method of claim 1 wherein the pre-positioned survival resources further comprise at least one of:
shelter;
clothing;
environmentally borne threat abatement;
medical facilities.

4. The method of claim 1 wherein the civilly-catastrophic event comprises an event that substantially disrupts society's infrastructure and ability to provide at least one life-sustaining resource.

5. The method of claim 4 wherein the civilly-catastrophic event is one that is likely to persist in substantial form for more than a predetermined period of time.

6. The method of claim 4 wherein the civilly-catastrophic event comprises at least one of:
a natural disaster;
a non-naturally-caused disaster.

7. The method of claim 6 wherein the natural disaster comprises at least one of:
a severe weather event;
a severe geophysical event;
a severe astronomical event;
a severe disease-based event;
a severe natural shortage of a life-sustaining resource.

8. The method of claim 6 wherein the non-naturally-caused disaster comprises at least one of:
an intentional act of aggression;
an unintentional act of aggression;
an unintended event that results in public dispersal of a severe environmentally borne danger.

9. The method of claim 1 wherein the first party provides private civil defense services to parties other than the authorized beneficiaries.

10. The method of claim 9 wherein the private civil defense services comprise at least one of:
a private civil defense shelter;
private civil defense survival supplies;
private civil defense transport service;
private civil defense rescue service;
privately developed customized instructions regarding appropriate survival actions to take in response to a civilly-catastrophic event.

11. The method of claim 1 wherein the second party comprises at least one of:
an insurance underwriter;
a financial products institution;
an insurance broker;
a bank;
a credit union;
an academic institution;
a club;
a certified financial planner;
a licensed securities dealer;
a religious organization;
a trade organization;
a fraternal organization.

12. The method of claim 1 further comprising, prior to an applicable civilly-catastrophic event:
providing to the authorized beneficiary a key to permit the authorized beneficiary to access the pre-positioned survival resources during an authorized time of need.

13. The method of claim 12 wherein the key comprises at least one of:
an identification card;
a code;
instructions regarding presentation of a biometric as pertains to the authorized beneficiary;
a physical key;
confirmation regarding acceptance of a code proffered by the authorized beneficiary.

14. The method of claim 1 further comprising, prior to an applicable civilly-catastrophic event:
permitting a third party other than the first party and the second party to interact with candidate policy holders and to arrange for candidate policy holders to become policy holders.

15. The method of claim 14 wherein permitting a third party other than the first party and the second party to interact with candidate policy holders and to arrange for candidate policy holders to become policy holders further comprises providing at least some of the premium to the third party.

16. The method of claim 15 wherein providing at least some of the premium to the third party comprises:
providing a first relative portion of the premium to the third party with respect to a first term of a policy for a given one of the policy holders;
providing a second relative portion of the premium to the third party with respect to a term of the policy that is subsequent to the first term for the given one of the policy holders, wherein the second relative portion is different than the first relative portion.

17. The method of claim 16 wherein the second relative portion is less than the first relative portion.

18. The method of claim 15 wherein providing at least some of the premium to the third party comprises:
providing a first relative portion of the premium to the third party with respect to a first term of a policy for a given one of the policy holders;
providing differing relative portions of the premium to the third party with respect to at least some terms of the policy that are subsequent to the first term for the given one of the policy holders, such that at least two of the differing relative portions of the premium are different from one another and from the first relative portion.

19. The method of claim 1 wherein permitting the authorized beneficiary to access the pre-positioned survival resources further comprises first determining that the authorized beneficiary is presently authorized to access the pre-positioned survival resources.

20. The method of claim 1 wherein permitting the authorized beneficiary to access the pre-positioned survival resources further comprises monitoring corresponding circumstances as are relevant to a need for the authorized beneficiary to continue to have access to the pre-positioned survival resources.

21. The method of claim 20 wherein the corresponding circumstances comprise at least one of:
persistence of survival-challenging conditions;
worsening of survival-challenging conditions;
abatement of survival-challenging conditions;
availability of publicly-sponsored survival assistance;
limitations as delimited by the policy as pertains to the authorized beneficiary;
observance of rules as have been established by the first party;
declarations regarding disasterhood by applicable governmental authorities;
a substantial lack of effective governmental influence with respect to an availability of survival requirements;
a total number of people who are substantially adversely directly affected by the civilly-catastrophic event within an occurrence zone of the civilly-catastrophic event;
confirmation by at least one of the first and second party that an applicable civilly-catastrophic event has occurred.

22. The method of claim 1 wherein compensating the first party with respect to permitting the authorized beneficiary to access the pre-provisioned survival resources further comprises the first party providing notice to the second party regarding the authorized beneficiary's access to the pre-provisioned survival supplies.

23. The method of claim 1 wherein the second party is partially, but not fully, owned by the first party.

24. The method of claim 1 further comprising:
receiving an application from a candidate policy holder to subscribe to one of the premium-based private civil security policies.

25. The method of claim 24 wherein receiving an application from a candidate policy holder to subscribe to one of the premium-based private civil security policies comprises receiving the application at a third party that is neither the first party nor the second party and who is authorized to solicit submissions of such applications.

26. The method of claim 24 further comprising:
assessing the application to determine whether to accept the application and provide the premium-based private civil security policy to the candidate policy holder.

27. The method of claim 26 wherein assessing the application comprises, at least in part, assessing a risk that the candidate policy holder will actually need to seek access to the pre-positioned survival resources during a coverage period when the policy provides corresponding private civil defense benefits.

28. The method of claim 26 wherein assessing the application to determine whether to accept the application and provide the premium-based private civil security policy to the candidate policy holder further comprises assessing the application to determine the premium to be required in exchange for providing the policy to the candidate policy holder.

29. The method of claim 28 wherein assessing the application to determine the premium to be required in exchange for providing the policy to the candidate policy holder further comprises making the assessment as a function, at least in part, of authorized beneficiary:
health;
age;
relative personal physical mobility;
survival training;
past behavior;
predicted future behavior.

30. The method of claim 1 wherein providing, via a first party, pre-positioned survival resources comprising at least hydration and nutritional consumables comprises providing a plurality of pre-positioned caches of the survival resources at corresponding geographically distal locations.

31. The method of claim 30 wherein permitting the authorized beneficiary to access the pre-positioned survival resources comprises permitting the authorized beneficiary to only access the pre-positioned survival resources as are located at a first one of the geographically distal locations.

32. The method of claim 30 wherein permitting the authorized beneficiary to access the pre-positioned survival resources comprises permitting the authorized beneficiary to access any of a plurality of the pre-positioned survival resources as are located at the geographically distal locations.

33. The method of claim 1 further comprising, prior to the applicable civilly-catastrophic event:
delivering to the first policy holder a short-term supply of survival resources comprising at least nutritional consumables.

34. The method of claim 33 wherein the short-term supply comprises a sufficient quantity of the survival resources to support survival of the first policy holder for at least a predetermined number of days.

35. The method of claim 34 wherein the predetermined number of days comprises about ten days.

36. The method of claim 33 wherein delivering to the first policy holder the short-term supply of supply resources further comprises permitting the first policy holder to purchase additional supply resources from the first party.

37. The method of claim 1 further comprising, prior to the applicable civilly-catastrophic event:
delivering a personal guide regarding specific personal actions to be taken by an authorized beneficiary of a policy as corresponds to the policy holder in response to a civilly-catastrophic event to thereby facilitate the authorized beneficiary taking appropriate helpful actions in such circumstances.

38. The method of claim 37 wherein delivering the personal guide comprises delivering the personal guide in response to receiving the premium.

39. The method of claim 37 wherein delivering the personal guide comprises delivering the personal guide in response to receiving consideration in excess of the premium.

40. A method of providing private civil defense benefits comprising:
at a first party and prior to an applicable civilly-catastrophic event:
offering premium-based private civil security policies with respect to providing civilly-catastrophic event-based access to pre-positioned survival resources that comprise, at least in part, hydration and nutritional consumables as are provided by a second party;
receiving a premium from a first policy holder to procure one of the policies for at least one corresponding authorized beneficiary;
at the first party and in response to an applicable civilly-catastrophic event that occurs during a period of coverage as corresponds to the policy that is procured by the first policy holder:
compensating the second party with respect to permitting the authorized beneficiary to access the pre-provisioned survival resources.

41. The method of claim 40 wherein the policies comprise at least one of:
time-limited rights of access;
event-limited rights of access;
inheritable rights of access;
rights of access predicated upon a series of periodic payments;
rights of access predicated upon a one-time payment;
ownership-based rights of access;
non-transferable rights of access;
transferable rights of access;
membership-based rights of access;
fractionally-based rights of access;
non-ownership-based rights of access;
option-based rights of access.

42. The method of claim 40 wherein the pre-positioned survival resources further comprise at least one of:
shelter;
clothing;
environmentally borne threat abatement;
medical facilities.

43. The method of claim 40 wherein the civilly-catastrophic event comprises an event that substantially disrupts society's infrastructure and ability to provide at least one life-sustaining resource.

44. The method of claim 43 wherein the civilly-catastrophic event is one that is likely to persist in substantial form for more than a predetermined period of time.

45. The method of claim 43 wherein the civilly-catastrophic event comprises at least one of:
a natural disaster;
a non-naturally-caused disaster.

46. The method of claim 45 wherein the natural disaster comprises at least one of:
a severe weather event;
a severe geophysical event;
a severe astronomical event;
a severe disease-based event;
a severe natural shortage of a life-sustaining resource.

47. The method of claim 45 wherein the non-naturally-caused disaster comprises at least one of:
an intentional act of aggression;
an unintentional act of aggression;
an unintended event that results in public dispersal of a severe environmentally borne danger.

48. The method of claim 40 wherein the second party also provides private civil defense services to parties other than the authorized beneficiaries.

49. The method of claim 48 wherein the private civil defense services comprise at least one of:
a private civil defense shelter;
private civil defense survival supplies;
private civil defense transport service;
private civil defense rescue service.

50. The method of claim 40 wherein the first party comprises at least one of:
an insurance underwriter;
a financial products institution;
an insurance broker;
a bank;
a credit union;
an academic institution;
a club.

51. The method of claim 40 further comprising, prior to an applicable civilly-catastrophic event:
providing to the authorized beneficiary a key to permit the authorized beneficiary to access the pre-positioned survival resources during an authorized time of need.

52. The method of claim 51 wherein the key comprises at least one of:
an identification card;
a code;
instructions regarding presentation of a biometric as pertains to the authorized beneficiary;
a physical key;
confirmation regarding acceptance of a code proffered by the authorized beneficiary.

53. The method of claim 40 further comprising, prior to an applicable civilly-catastrophic event:
permitting a third party other than the first party and the second party to interact with candidate policy holders as a broker to arrange for candidate policy holders to become policy holders.

54. The method of claim 53 wherein permitting a third party other than the first party and the second party to interact with candidate policy holders as a broker and to arrange for candidate policy holders to become policy holders further comprises providing at least some of the premium to the third party.

55. The method of claim 54 wherein providing at least some of the premium to the third party comprises:
providing a first relative portion of the premium to the third party with respect to a first term of a policy for a given one of the policy holders;
providing a second relative portion of the premium to the third party with respect to a term of the policy that is subsequent to the first term for the given one of the policy holders, wherein the second relative portion is different than the first relative portion.

56. The method of claim 55 wherein the second relative portion is less than the first relative portion.

57. The method of claim 40 further comprising determining when the authorized beneficiary is entitled to access the pre-positioned survival resources.

58. The method of claim 57 wherein determining when the authorized beneficiary is entitled to access the pre-positioned survival resources further comprises monitoring corresponding circumstances as are relevant to a need for the authorized beneficiary to continue to have access to the pre-positioned survival resources.

59. The method of claim 58 wherein the corresponding circumstances comprise at least one of:
persistence of survival-challenging conditions;
worsening of survival-challenging conditions;
abatement of survival-challenging conditions;
availability of publicly-sponsored survival assistance;
limitations as delimited by the policy as pertains to the authorized beneficiary;
observance of rules as have been established by the first party;
declarations regarding disasterhood by applicable governmental authorities;
a substantial lack of effective governmental influence with respect to an availability of survival requirements;
a total number of people who are substantially adversely directly affected by the civilly-catastrophic event within an occurrence zone of the civilly-catastrophic event.

60. The method of claim 40 wherein compensating the second party with respect to permitting the authorized beneficiary to access the pre-provisioned survival resources further comprises receiving notice from the second party regarding the authorized beneficiary's access to the pre-provisioned survival supplies.

61. The method of claim 40 wherein the first party is partially, but not fully, owned by the second party.

62. The method of claim 40 further comprising:
receiving an application from a candidate policy holder to subscribe to one of the premium-based private civil security policies.

63. The method of claim 62 wherein receiving an application from a candidate policy holder to subscribe to one of the premium-based private civil security policies comprises receiving the application at a third party that is neither the first party nor the second party and who is authorized to solicit submissions of such applications.

64. The method of claim 62 further comprising:
assessing the application to determine whether to accept the application and provide the premium-based private civil security policy to the candidate policy holder.

65. The method of claim 64 wherein assessing the application comprises, at least in part, assessing a risk that the candidate policy holder will actually need to seek access to the pre-positioned survival resources during a coverage period when the policy provides corresponding private civil defense benefits.

66. The method of claim 64 wherein assessing the application to determine whether to accept the application and provide the premium-based private civil security policy to the candidate policy holder further comprises assessing the application to determine the premium to be required in exchange for providing the policy to the candidate policy holder.

67. The method of claim 66 wherein assessing the application to determine the premium to be required in exchange for providing the policy to the candidate policy holder further comprises making the assessment as a function, at least in part, of authorized beneficiary:
health;
age;
physical condition;
relative personal physical mobility;
survival training;
past behavior;
predicted future behavior.
